(19) United States
(12) Patent Application Publication
(10) Pub. No.: US 2010/0198222 A1
(43) Pub. Date: Aug. 5, 2010

(54) Rongeur

(76) Inventor: James A. Schneiter, Lake Forest, IL (US)

Correspondence Address: BRINKS HOFER GILSON & LIONE, P.O. BOX 10395, CHICAGO, IL, 60610, US

(21) Appl. No.: 12/798,527

(22) Filed: Apr. 6, 2010

Related U.S. Application Data
(63) Continuation-in-part of application No. 11/529,696, filed on Sep. 26, 2006, now Pat. No. 7,691,107.
(60) Provisional application No. 60/722,484, filed on Sep. 30, 2005.

(51) Int. Cl. A61B 17/00

(52) U.S. Cl. 606/83

(57) ABSTRACT

A rongeur has a fixed shank and a moveable crossbar. The crossbar is connected to and axially aligned with the fixed shank. The crossbar has a distal end with a cutting portion located therein. A port is connected to one of the fixed shank and the moveable crossbar. An access channel is connected to the port and provides improved flushing, cleaning, sterilization, drying and/or lubricating capabilities.

### RELATED APPLICATIONS

This application is a continuation-in-part of prior application Ser. No. 11/529,696, filed Sep. 26, 2006, now U.S. Pat. No. 7,691,107, which claims the benefit of U.S. Provisional Application No. 60/722,484, filed Sep. 30, 2005. The entire contents of both of these documents are incorporated herein by reference, except that in the event of any inconsistent disclosure or definition from the present specification, the disclosure or definition herein shall be deemed to prevail.

### TECHNICAL FIELD

The present teachings relate generally to the field of medical instruments and, more particularly, to rongeurs.

### BACKGROUND

The inadequate or improper cleaning and sterilization of surgical instruments has been documented by the Center for Disease Control (CDC) to be a major cause of surgical site infections (SSIs). According to data from the CDC, SSIs are the cause of over 30,000 deaths annually in the United States alone. A CDC publication entitled “Guideline for Prevention of Surgical Site Infection” states that SSIs are particularly troublesome because “one third involved organs and spaces accessed during the operation.” This CDC publication further states that “[when] surgical patients with SSIs died, 77 percent of the deaths were reported to be related to the infection and the majority, 93 percent, were serious infections involving organs and spaces accessed during the operation.” In spite of “[a]dvances in infection control practices [that] include improved operating room ventilation, sterilization methods and barriers, surgical techniques and availability of antimicrobial prophylaxis,” the CDC observes that “SSIs remain a substantial cause of morbidity and mortality among hospitalized patients.”

One common source of SSIs is the use of surgical instruments that cannot be properly cleaned and/or sterilized on every reprocessing cycle. Entrapped bio-burden can inhibit the sterilization process, such that sterility of the instrument after re-processing cannot be guaranteed. In addition, unless the instrument is thoroughly dried after the sterilization cycle, waterborne pathogens can survive in inaccessible spaces and further result in a contaminated instrument. Use of a contaminated instrument creates an increased risk of a surgical infection to the patient.

One surgical instrument particularly susceptible to the above-mentioned problems related to improper cleaning and/or sterilization is the rongeur. The rongeur is a hand-held device used to remove small amounts of bodily material during surgery (e.g., neck surgery, spinal surgery, and the like). During normal operation of the instrument, blood, mucous, tissue, bone chips and/or additional bio-burden typically become trapped within the instrument.

In attempting to solve the problem of bio-burden and/or waterborne pathogens collecting on the internal interfacing surfaces of rongeurs, several styles of rongeurs have been developed that can be taken apart to expose all surfaces to the cleaning process. By way of example, U.S. Pat. No. 6,638,280 B2 to Agbodoe describes a rongeur that is intended to be taken apart to expose all of its surfaces to cleaning (e.g., col. 4, lines 4-6). However, such a “take apart” instrument has a number of drawbacks, as described below.

First, surgeons frequently complain that the tactile feel and response of a “take apart” instrument is inferior to that of a conventional, “non-take apart” instrument, thus making the instrument more difficult to use during neurosurgical procedures. Second, even with well-trained and educated reprocessing personnel, it is difficult and time-consuming to disassemble, decontaminate, clean, and reassemble a “take-apart” instrument—not to mention the associated reprocessing concerns that include loss/misplacement of parts and improper instrument reassemblies. Third, reprocessing personnel who fail to recognize an instrument as being of the “take apart” variety, or who lack the training and/or tools to properly disassemble, clean, and reassemble the instrument, may skip the entire decontamination and cleaning process, which results in a contaminated instrument being returned to the operating room with the attendant risk of surgical infection. Fourth, infection-causing waterborne pathogens can remain within an instrument if it is not properly dried after cleaning and sterilizing (e.g., within the drainage holes 88 of the device described in U.S. Pat. No. 6,638,280 B2). Fifth, it is very difficult to properly lubricate the instrument without completely disassembling and then reassembling it, and the lack of proper lubrication inside the instrument causes galling of the metal making it difficult for a surgeon to open and close the instrument during neurosurgical procedures, thereby significantly shortening the life of the instrument.

In short, there are serious drawbacks to the use of “take apart” instruments as described above. While other approaches have been suggested that involve the use of more complicated instruments, these other approaches create new levels of manufacturing and user complexity.

### SUMMARY

The scope of the present invention is defined solely by the appended claims, and is not affected to any degree by the statements within this summary.

A rongeur includes (a) a fixed shank having a first interfacing surface and a distal end that includes a footplate; (b) a moveable crossbar that is connected to and axially aligned with the fixed shank, wherein the crossbar includes a distal end with a cutting portion located therein and a second interfacing surface configured for mating with the first interfacing surface along an interface therebetween; (c) a port connected to one of the fixed shank and the moveable crossbar, wherein the port is located in a proximal end of one of the fixed shank and the crossbar; and (d) an access channel connected to the port and configured for providing fluid access to an interior of the rongeur. The access channel extends axially and longitudinally along the interface between the fixed shank and the moveable crossbar and opens adjacent the cutting portion of the crossbar. One of the crossbar and the fixed shank includes a rail and the other of the crossbar and the fixed shank includes a receiving channel configured for receiving the rail. At least a portion of the rail and/or the receiving channel forms a portion of the access channel.

### BRIEF DESCRIPTION OF THE DRAWINGS

FIG. 1 is a perspective view of a first rongeur embodying features of the present invention with its crossbar shown in an open or cutting position.

FIG. 2 is a top plan view of the distal end of the rongeur in FIG. 1.

FIG. 3 is a side plan view of the rongeur in FIG. 1 with the port and access channel illustrated in dashed lines.

FIG. 4 is a cross-sectional view of the rongeur in FIG. 1 taken along the line 4-4 and showing an embodiment of an access channel.

FIG. 5 is a cross-sectional view of the rongeur in FIG. 1 taken along the line 5-5 and showing an embodiment of a retaining rail and receiving channel.

FIG. 5A is a cross-sectional view of a rongeur showing an alternative embodiment of a retaining rail and receiving channel

FIGS. 6A-6F show additional embodiments of an access channel passing through at least one of the crossbar and fixed shank.

FIG. 7 is a perspective view of a second rongeur embodying features of the present invention.

### DETAILED DESCRIPTION

By way of introduction, rongeurs having improved flushing, cleaning, sterilization, drying, and/or lubricating capabilities have been discovered and are described hereinbelow. Although embodiments described herein relate generally to rongeurs, the present teachings are also applicable to other types of medical instruments—particularly those that have poorly accessible and/or inaccessible surfaces that are difficult to properly flush, clean, sterilize, dry and/or lubricate.

As used herein, the phrase “body material” is intended broadly to encompass any type of biological material or bio-burden including but not limited to bone, tissue, cartilage, blood, and the like, and combinations thereof.

In addition, the phrase “access channel” is intended broadly to encompass any passageway in an instrument, regardless of its size or shape, which is operative to perform cleaning, flushing, sterilization, drying and/or lubricating of the instrument.

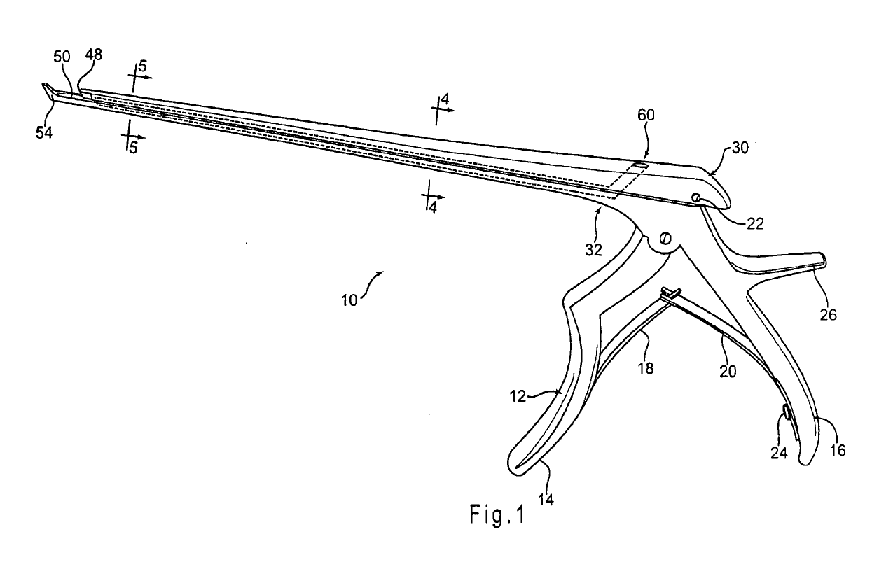

FIG. 1 is an illustration of an embodiment of a rongeur 10 shown in an open or cutting position. The rongeur 10 includes a handle 12 having a front grip 14 and a rear grip 16. Spring elements 18 and 20 force apart the front grip 14 and the rear grip 16. The front grip 14 and rear grip 16 are pivotably interconnected by means of a screw 22. Two additional screws (one shown) 24 are used to secure the spring elements 18 and 20 to the front grip 14 and the rear grip 16. The rear grip 16 includes a thumb portion 26 that accommodates the thumb of a surgeon. A crossbar 30 is slideably positioned over a fixed shank 32. The crossbar 30 moves in a reciprocating plane that is axially aligned with respect to the fixed shank 32.

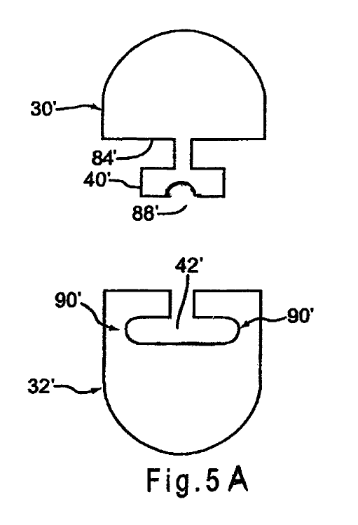

As best shown in FIG. 5, the crossbar 30 is slidably connected to the fixed shank by means of a rail 40 and a receiving channel 42. As further shown in FIG. 5, the rail 40 projects downwardly from the crossbar 30 and is slideably received within the channel 42. In some embodiments, the receiving channel 42 forms a portion of the access channel that is described herein.

In some embodiments, materials such as stainless steel and/or tungsten carbide are used to form the rongeur 10. Such materials may optionally be micro-polished to decrease the ability of bio-burden to adhere to the surface of the instrument, increase the effectiveness of the detergent flush in removing bio-burden, increase bacterial kill rates during the sterilization cycle, increase the volume of moisture removed during the drying cycle, increase the effectiveness of the lubrication process, improve the overall function and operation of the instrument, and extend the useful life of the instrument.

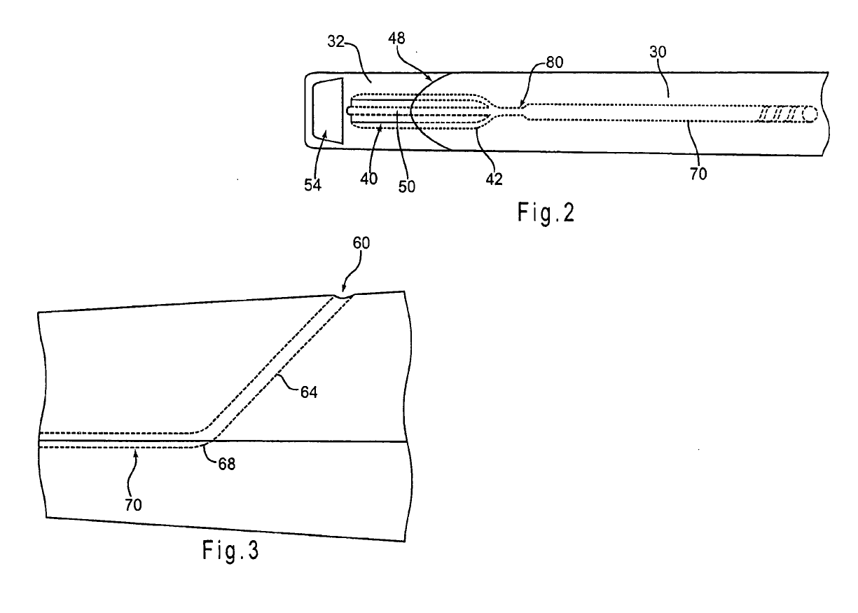

As shown in FIG. 1, the front grip 14 is connected to the crossbar 30 such that when it is squeezed, the crossbar 30 is driven forward. As shown in FIG. 2, the crossbar 30 includes a distal ending having a cutting surface 48 adjacent a bite opening 50. A footplate 54 is located at the distal end of the fixed shank 32. As shown in FIGS. 1-2, the spring elements 18 and 22 hold the cutting surface 48 in the open position or spaced apart from the footplate 54. The cutting surface 48 and the footplate 54 can include a recess that retains the severed material.

During surgery, the surgeon squeezes or compresses the front grip 14 when the bite opening 50 is adjacent the desired body material. By squeezing the front grip 14 toward the rear grip 16, the cutting surface 48 is drawn toward the footplate 54. When the cutting surface 48 contacts the tissue, it “bites” the body material or tissue to remove a specimen for testing. When the surgeon releases the pressure on the front grip 14 and the rear grip 16, the spring elements 18 and 20 force the front grip 14 and the rear grip 16 apart, thereby sliding the crossbar 30, and particularly cutting surface 48, away from the footplate 54. This is the open position illustrated in FIG. 1 and the position from which the specimen can be removed.

One embodiment of a port 60 and the access channel 42 is illustrated in FIGS. 1-5. As shown in FIG. 3, the port 60 is located in the crossbar 30. In some embodiments, the port 60 has a diameter of 0.080″. The access channel 42 has an angled throat portion 64. In some embodiments, the throat portion 64 has a diameter that tapers down to 0.050″ at the proximal end 68. The taper of the throat portion 64 provides for a compression fit between the wall of the throat and an infusion cannula that is inserted into the port 60 for distributing the flushing solution into the access channel 42. The taper of the throat portion 64 also increases the velocity of the cleaning solution to thereby provide a more effective cleaning.

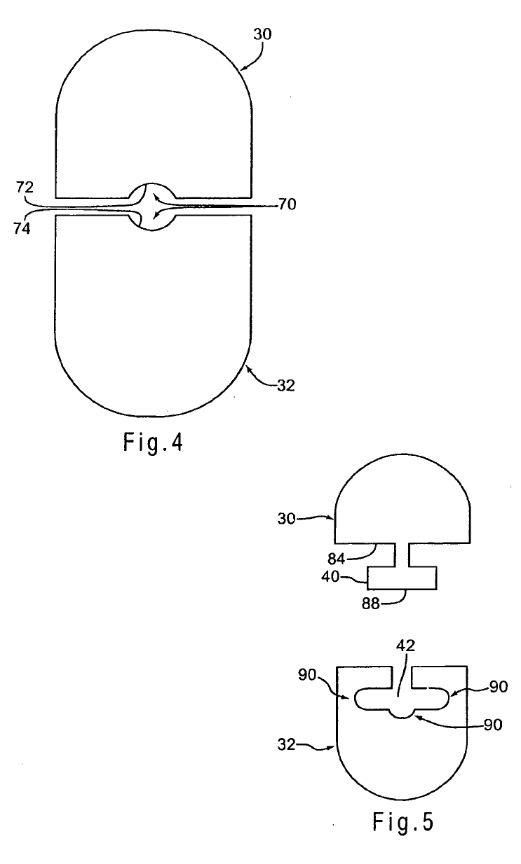

FIG. 4 shows an embodiment of an access channel 42 with an axial portion 70 shown in cross-section. The axial portion 70 includes a curved upper portion 72 in the crossbar 30 and a curved lower portion 74 in the fixed shank 32. As shown in FIG. 2, the axial portion 70 has a narrowed throat portion 80 at the distal end. In some embodiments, the throat portion 80 has a reduced diameter of 0.040″ that serves to increase the venturi effect of any cleaning solution passing therethrough.

Adjacent the throat portion 80, the rail 40 projects downwardly from the crossbar 30 into the receiving channel 42. In some embodiments, as shown in FIG. 5, the rail 40 has an I-beam shape with cutout portions 84 and a flat bottom surface 88. In some embodiments, the receiving channel 42 has an inverted cross-like shape that is sized to accommodate the rail 40. The receiving channel 42 has curved, cornerless portions 90 that allow for cleaning solution to pass along the surfaces of the rail 40 to remove trapped bio-burden, permit the steam to reach these surfaces during the sterilization cycle to increase bacterial kill rates, void residual moisture from these surfaces during the drying cycle to decrease the risk of waterborne pathogens contaminating the instrument, and to permit proper lubrication of the instrument.

FIG. 5A shows an alternative configuration of a rail and receiving channel that can be used in place of the arrangement shown in FIG. 5. In this alternative configuration, a rail 40′ projects downwardly from a crossbar 30′ into a receiving channel 42′ in a fixed shank 32′. The rail 40′ has cutout portions 84′ and a bottom surface 88′ having a curved depression therein. The receiving channel 42′ is sized to accommodate the rail 40′ and has curved, cornerless portions 90′. In a manner analogous to that described above in connection with the embodiment shown in FIG. 5, the alternative embodiment shown in FIG. 5A allows cleaning solution to pass along the surfaces of the rail 40′ to remove trapped bio-burden, permit the steam to reach these surfaces during the sterilization cycle to increase bacterial kill rates, void residual moisture from these surfaces during the drying cycle to decrease the risk of waterborne pathogens contaminating the instrument, and to permit proper lubrication of the instrument.

Conventional cleaning solutions such as enzymatic detergents or any other approved medical device cleaning solution may be used. The cleaning solutions are inserted through the port 60 into the access channel 70 using a cannula attached to an infusion device such as a syringe in order to flush and clean the rongeur 10. In particular, the crossbar 30 and the fixed shank 32 are exposed to body material such as tissues or fluids that can collect within the space between the crossbar 30 and the fixed shank 32. In contrast to previous rongeurs, the access channel 70 provides a means for cleaning the space between the interfacing surfaces of the crossbar 30 and the fixed shank 32 and the area around the rail 40. The access channel 70 serves as a conduit for the steam to penetrate the interior of the instrument during the sterilization cycle, thereby increasing bacteria kill and reducing the risk of a contaminated instrument infecting a patient.

In some embodiments, the access channel 70 may be used for improved drying of the rongeur 10. During the sterilization process, moisture collects in the internal recesses of the instrument. This residual moisture can contain waterborne pathogens that result in a contaminated instrument after the sterilization cycle. The access channel 70 provides a means to void the instrument of residual moisture during the drying cycle and minimize the risk of waterborne pathogens remaining inside the instrument.

In some embodiments, conventional lubrication solutions or materials may be used to properly lubricate the rongeur 10. The lubricant is inserted through the port 60 into the access channel 70 using a cannula attached to an infusion device such as a syringe in order to lubricate the internal surfaces of the rongeur 10. The access channel 70 provides a means for lubricating the space between the interfacing surfaces of the crossbar 30 and the fixed shank 32 and the area around the rail 40. The access channel 70 provides a conduit to infuse an instrument lubricant into the instrument to reduce galling, improve instrument performance and extend the useful life of the instrument without having to go through the time-consuming, expensive process of disassembling, lubricating, and then reassembling the instrument.

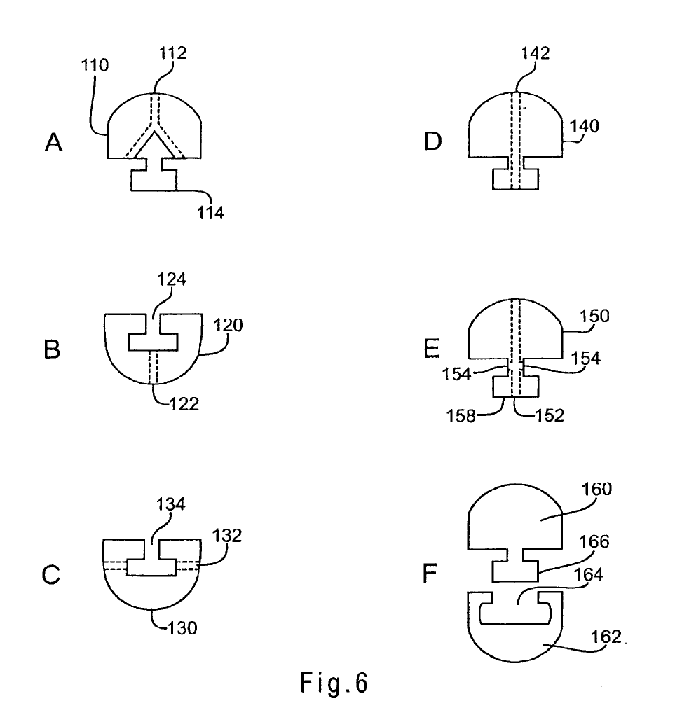

FIGS. 6A-F illustrate additional embodiments in accordance with the present teachings. In FIG. 6A, a crossbar 110 includes an access channel 112 having an inverted Y-shaped configuration adjacent a rail 114. FIG. 6B illustrates a fixed shank 120 having an access channel 122 extending downwardly from a receiving channel 124. FIG. 6C illustrates a fixed shank 130 similar to that shown in FIG. 6B except that an access channel 132 extends outwardly from a receiving channel 134. FIGS. 6D and 6E illustrate additional configurations of crossbars 140 and 150, respectively. The crossbar 140 of FIG. 6D includes an access channel 142 that extends downwardly through the center thereof. The crossbar 150 of FIG. 6E includes a similar access channel 152 with two additional side openings 154 extending through a portion of the rail 158. In FIG. 6F, a crossbar 160 and a fixed shank 162 include an alternate form of a receiving channel 164 that receives a rail 166. In some embodiments, a port 60 and an access channel 70, such as illustrated in FIGS. 1-5, are included in the configurations described above.

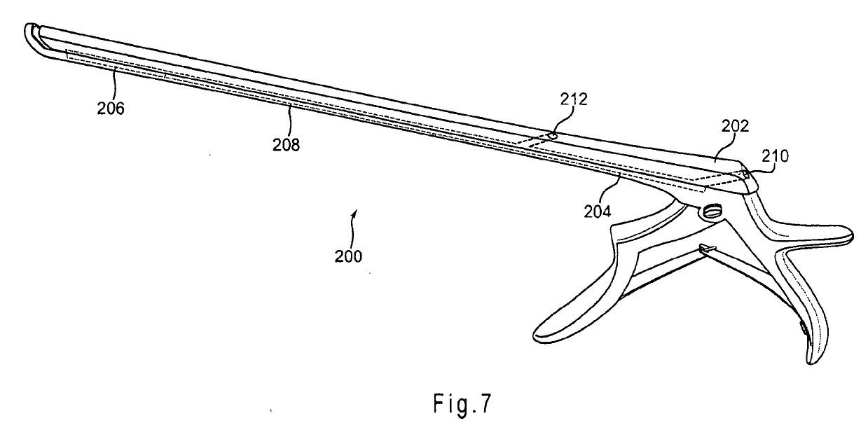

FIG. 7 illustrates another embodiment of a rongeur 200 that is similar to embodiments shown in FIGS. 1-5 except that a crossbar 202 includes two spaced apart rail portions 204 and 206 that are received by a receiving channel 208. The rongeur 200 also includes two ports 210 and 212 to provide fluid access to the receiving channel 208.

The following examples and representative procedures illustrate features in accordance with the present teachings, and are provided solely by way of illustration. They are not intended to limit the scope of the appended claims or their equivalents.

### EXAMPLES

General Comments: To evaluate cleaning efficacies of rongeurs embodying features of the present invention, the devices were contaminated with defibrinated blood soil (DBLSO) containing Geobacillus stearothermophilus (ATCC 7953). Bio-burden extractions were performed to determine the number of viable organisms present on one positive control device. Three test devices were cleaned and additional bio-burden assays were performed to determine the bio-load reduction of each device.

Culture Preparation: The test soil was inoculated with the test organism from a stock spore suspension maintained at 2-8° C. to yield a minimum population of 10⁴ CFU/mL. A standard plate count was performed on the inoculated test soil to determine the initial titer of the test organism.

Sample Contamination: Approximately 2 inches of the distal ends of the devices were immersed in the inoculated test soil. The remaining test soil was placed into a clean spray bottle, and the rest of the devices were sprayed with the test soil to achieve even coverage. The devices were simulated by opening and closing the jaws 10 times for each device. The devices were then allowed to remain in contact with the test soil for 15 minutes with the jaws open. The soiled devices were then placed into a clean pan, and the pan was covered with a towel dampened with purified water (PURW) and allowed to set for 30 minutes to simulate the wait time between contamination and cleaning.

Positive Control Recovery: The positive control device was tested using the bio-burden method described below.

Cleaning Procedure: The enzymatic detergent sold under the tradename ENZOL was prepared following manufacturer's recommendations using lukewarm tap water. Each device was fully immersed in the prepared detergent and allowed to soak for a minimum of 5 minutes with the jaws open. Each device was thoroughly cleaned with a soft bristle brush until all visible soil had been removed with particular attention given to crevices and other difficult-to-clean areas of the device. Using a flush cannula that fit tightly into the flush port before flushing, the flush port on each device was flushed three times. Following cleaning, the devices were rinsed in lukewarm tap water for a minimum of one minute. The flush cannula was used to aid in rinsing each device three times each.

The enzymatic detergent sold under the tradename ENZOL was prepared following manufacturer's recommendations using lukewarm tap water in an ultrasonic cleaning unit. The flush cannula and each device (with the jaws open) were fully immersed in the prepared detergent and allowed to sonicate for a minimum of 10 minutes. Following sonication, the devices were rinsed in lukewarm tap water for a minimum of one minute. The sonicated flush cannula was used to aid in rinsing the devices. The devices were dried using a clean soft cloth. Pressurized air was also used to aid in drying, specifically the flush port of the device.

Bio-burden Testing: The positive control and cleaned devices were tested for bio-burden by immersion in the peptone sold under the tradename TWEEN and manual shaking (100 times) to extract the organisms present. Aliquots of the extract fluid were diluted where appropriate and plated onto soybean casein digest agar (SCDA) or filtered through a 0.45 μm membrane and the membrane was placed onto SCDA. Plates were incubated at 55-60° C. for 24±3 hours and colonies were enumerated.

Calculations: The percent reduction after cleaning was calculated using the following formula:

$$\% reduction = 100 - ( \frac{final population}{initial population} \times 100 )$$

The log reduction after cleaning was calculated using the following formula:

log reduction=log initial population−log final population

wherein the initial population represents positive control titer and wherein the final population represents recovered counts of each device.

Results: Each device was individually inoculated and some variability is expected between devices. The cleaned devices were free of visible soil. Results of the cleaning evaluation are shown in Table 1. As shown by these data, the bio-burden present in a dirty rongeur in accordance with the present invention can be reduced by greater than 99.99980% after cleaning! As these dramatic data show—surprisingly and unexpectedly—a rongeur in accordance with the present invention can be cleaned extremely effectively.

TABLE 1
Cleaning Results

| DEVICE | COUNTS | PERCENT | LOG₁₀ |
|---|---|---|---|
| IDENTIFICATION | PER UNIT | REDUCTION (%) | REDUCTION |
| 1 | <1.0 × 10⁰ | >99.99980 | >5.7 |
| 2 | <1.0 × 10⁰ | >99.99980 | >5.7 |
| 3 | <1.0 × 10⁰ | >99.99980 | >5.7 |

DBLSO Titer: 1.4 × 10⁵ CFU/mL
Positive Control Titer: 5.1 × 10⁵ CFU/device

The foregoing detailed description and accompanying drawings have been provided by way of explanation and illustration, and are not intended to limit the scope of the appended claims. Many variations in the presently preferred embodiments illustrated herein will be apparent to one of ordinary skill in the art, and remain within the scope of the appended claims and their equivalents.

1. A rongeur, comprising:
a) a fixed shank comprising a first interfacing surface and a distal end comprising a footplate;
b) a moveable crossbar that is connected to and axially aligned with the fixed shank, wherein the crossbar comprises a distal end with a cutting portion located therein and a second interfacing surface configured for mating with the first interfacing surface along an interface therebetween;
c) a port connected to one of the fixed shank and the moveable crossbar, wherein the port is located in a proximal end of one of the fixed shank and the crossbar; and
d) an access channel connected to the port and configured for providing fluid access to an interior of the rongeur, wherein the access channel extends axially and longitudinally along the interface between the fixed shank and the moveable crossbar and opens adjacent the cutting portion of the crossbar;
wherein one of the crossbar and the fixed shank comprises a rail and the other of the crossbar and the fixed shank comprises a receiving channel configured for receiving the rail; and
wherein at least a portion of the rail and/or the receiving channel forms a portion of the access channel.

2. The invention of claim 1 wherein the port and the access channel provide for one or a plurality of improved flushing, cleaning, sterilizing and lubricating of the first and second interfacing surfaces.

3. The invention of claim 1 wherein the first interfacing surface and the second interfacing surface are micro-polished.

4. The invention of claim 3 wherein the port and the access channel provide for one or a plurality of improved flushing, cleaning, sterilizing and lubricating of the first and second interfacing surfaces.

5. The invention of claim 1 wherein the access channel is located in the fixed shank.

6. The invention of claim 1 wherein the port and the access channel are located in the crossbar.

7. The invention of claim 1 wherein the port is located in the crossbar and the access channel passes within both the crossbar and the fixed shank.

8. The invention of claim 7 wherein the access channel comprises a top curved portion passing axially along the crossbar and a bottom curved portion passing axially along the fixed shank.

9. The invention of claim 1 wherein the access channel comprises more than one fluid flow path.

10. The invention of claim 1 wherein the crossbar comprises the rail and the fixed shank comprises the receiving channel.

11. The invention of claim 1 wherein the crossbar comprises the receiving channel and the fixed shank comprises the rail.

12. The invention of claim 10 wherein the rail comprises an I-beam shape having a substantially flat bottom surface.

13. The invention of claim 12 wherein the receiving channel comprises an inverted cross-like shape sized to accommodate the rail.

14. The invention of claim 10 wherein the rail comprises a curved depression in a bottom surface thereof.

15. The invention of claim 14 wherein the receiving channel comprises a substantially flat bottom surface and is sized to accommodate the rail.
